(19) United States
(12) Patent Application Publication
(10) Pub. No.: US 2008/0125762 A1
(43) Pub. Date: May 29, 2008

(54) Grip Element for a Surgical Instrument

(75) Inventor: Jurgen Hiller, Dettingen (DE)

Correspondence Address: DICKSTEIN SHAPIRO LLP, 1825 EYE STREET NW, Washington, DC, 20006-5403, US

(73) Assignee: ERBE Elektromedizin Gmbh, Tubingen (DE)

(21) Appl. No.: 11/568,407

(22) PCT Filed: May 12, 2005
(86) PCT No.: PCT/EP05/05197

§ 371 Date: Sep. 21, 2007

(30) Foreign Application Priority Data
May 18, 2004 (DE) 10 2004 024 658.0
Jun. 22, 2004 (DE) 10 2004 030 030.5

(51) Int. Cl. A61B 17/00; A45C 13/22

(52) U.S. Cl. 606/1; 16/430

(57) ABSTRACT

The present invention relates to a grip element (2) for a surgical instrument, in particular for a tubular-shaft instrument, that comprises a handle (4) and an instrument holder (8) adapted to the surgical instrument. The instrument holder can be made integral with the surgical instrument or comprise a receptacle (18) for holding the surgical instrument. The grip element (2) is intended to enable the surgeon's hand and arm to be comfortable in a variety of operating positions. The grip element (2) is characterized by the fact that the handle (4) can be rotated and/or linearly shifted relative to the instrument holder (8).

### RELATED U.S. APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### FIELD OF THE INVENTION

The invention relates to a grip element for a surgical instrument, in particular for a surgical tubular-shaft instrument. The grip element comprises a handle and an instrument holder adapted to the surgical instrument.

### BACKGROUND OF THE INVENTION

Grip elements of this kind are in general use as a means of holding surgical instruments, in particular surgical tubular-shaft instruments. It is also known to construct grip elements in such a way that the surgical instrument can be operated by way of the grip element.

Tubular-shaft instruments are special surgical instruments employed in particular for endoscopic surgery. They are designed to be guided through a narrow opening in the body and inserted as far as a target site within the body cavity, so as to carry out a desired action at the target site, such as clamping off, cutting, extracting a tissue sample or stanching blood flow. In accordance with this application, an elongated intermediate piece is disposed between a tool and the grip element. By way of the grip element the tubular-shaft instrument can be positioned and operated. It is also known to attach electrical connectors to the grip element and guide appropriate electrical leads through the intermediate piece so that a voltage, in particular a high-frequency alternating voltage, can be applied to the tool.

The documents cited below show that a large number of different surgical instruments are known. The present invention is not concerned with the particular design of a tool, an intermediate piece, electrical connectors or a mechanical coupling between a component on the grip element for operating the tool and the tool itself. Accordingly, these features are not discussed in detail here.

The document U.S. Pat. No. 5,250,073 relates to a tubular-shaft instrument in the form of a biopsy forceps, which comprises a handle, an elongated hollow body and, at the distal end of the tubular-shaft instrument, a tool in the form of forceps jaws. The elongated hollow body extends between a distal end of the handle and the jaws of the forceps. Another tubular-shaft instrument with a handle is known from WO 94 05223. The arrangement of the handle relative to the remaining parts of the tubular-shaft instrument is unalterable in the case of both U.S. Pat. No. 5,250,073 and WO 94 05223.

Other tubular-shaft instruments with fixedly disposed handles are known from U.S. Pat. No. 5,569,243, U.S. Pat. No. 5,391,166 and U.S. Pat. No. 5,545,170.

U.S. Pat. No. 5,258,006 describes a bipolar tubular-shaft instrument with a handle, a tool in the form of forceps at the distal end of the tubular-shaft instrument, an outer tube and an inner tube passing through the outer tube. The handle is fixed in position relative to the outer tube. By actuating an actuation element, which preferably is constructed as a pivoted lever, the inner tube is displaced relative to the outer tube so as to bring about opening or closing of the jaws of the forceps. Electrical leads are also provided, which are guided through the lumen of the inner tube and by way of which an alternating voltage can be applied between the two jaws. The tubular-shaft instrument comprises means for rotating the outer tube and the inner tube together with the electrical leads about an axis oriented parallel to the long axis of the tubular-shaft instrument. Such a rotary movement is preferably triggered by turning a knob that is disposed on the handle.

An apparatus for extracting tissue samples from sites deeper within the body is known from WO 96 06563. A flexible catheter section is disposed between a grip element and a cutting tool, the catheter section being designed for insertion into the body, with the cutting tool at its front end, through a suitable tubular sheath such as a vein, so that the cutting tool can be positioned at the desired site in the body for removal of the tissue sample. The cutting tool is triggered to snap together by way of the grip element. Because the catheter section is flexible, the hand can be held in a comfortable position during operation, but no stable arrangement between grip element and cutting tool can be achieved.

Furthermore, U.S. Pat. No. 2,427,873 describes an apparatus for the insertion of clamps that can be used to close skin incisions, opened arteries and the like. This apparatus comprises a gripping and operating element connected to the actual tool for inserting the clamps by way of a tubular intermediate piece. The tubular piece together with the tool end can be removed from the gripping and operating element, and can furthermore be rotated about an axis parallel to its long axis and immobilized when it is in a desired orientation with respect to the gripping and operating element.

The document DE 41 32 261 describes an ergonomic handle for surgical instruments in which two grip elements end in two eyes for the index and middle finger and can be moved relative to one another like scissors. To the upper grip element there is also attached a saucer-like structure to serve as a bearing surface for the thumb.

From DE 197 02 447 C2 a handle for a surgical tubular-shaft instrument is known, in which the grip element comprises a shaft holder with a shaft receptacle for the tubular shaft of the tubular-shaft instrument and a carrier element oriented at an angle to the long axis of the tubular shaft. A handle projects at an angle from the proximal end region of the carrier element, crosses the long axis of the tubular shaft and extends beyond it. In addition an actuating element, preferably in the form of a pivoted lever, is provided, being pivotably mounted on the grip element for actuation of a tool disposed at the distal end of the tubular shaft. Such a construction is intended to make it possible for the surgeon holding the instrument handle to bring his forearm into a position where it coincides with the long axis of the tubular shaft.

The previously known designs of a grip element do make it possible for the surgeon to grasp the grip element in different positions, partly owing to a special configuration and arrangement of the grip elements. Nevertheless, it is not always possible to adopt a comfortable hand and arm position. In particular when space is limited and the operation site is difficult to access, a bent and hence tiring hand position is often inevitable. During relatively long operations this can make it impossible to maintain relaxed holding and precisely targeted positioning, which has an adverse effect on the outcome of the operation.

### BRIEF SUMMARY OF THE INVENTION

Accordingly, it is the objective of the present invention to make available a grip element for surgical instruments that does not present the above-mentioned disadvantages and, in particular, makes it possible for the surgeon's hand and arm to be in a comfortable position.

This objective is achieved by a grip element according to Claim 1. Advantageous embodiments, as well as a surgical instrument in accordance with the invention and an advantageous application of a grip element in accordance with the invention, are evident in the subsequent claims.

The invention is based on the essential idea of conceiving a grip element that can be adapted to whatever situation exists in an operation, in particular to the spatial arrangement between the operation site and the surgeon. This idea is put into practice in that the grip element comprises a first part that can be or is connected to the surgical instrument, and a second part that is designed to be gripped and can be moved relative to the first part. Accordingly, a handle is provided on the grip element that can be rotated and/or moved linearly with respect to an instrument holder adapted to the surgical instrument.

The grip element in accordance with the invention thus allows the relative positions of handle and instrument holder, and hence the relative positions of handle and surgical instrument, to be adapted to the current spatial arrangement. The orientation of the handle, which can be adjusted on-site, makes possible an ergonomic hand position, even when a working end of the surgical instrument is to be guided to lateral operation sites and/or those otherwise difficult to reach.

The grip element can be used for a large number of surgical instruments, but in particular for tubular-shaft instruments, regardless of the particular tool disposed thereon and how it is to be actuated. Regarding the configuration of the handle and the instrument holder, likewise, known and well-proven shapes and arrangements can be within its scope, insofar as they permit rotation and/or linear movement of the handle relative to the instrument holder.

The grip element preferably makes it possible for the surgical instrument also to be held at an angle to the forearm of the surgeon with no need for the wrist to be bent to the side. The directional terminology used here is based on the idea that a grip-element plane, in which the elongated handle and the instrument holder predominantly extend, is vertically oriented. Moreover, when reference is made to the orientation of the surgical instrument, it should be kept in mind that surgical instruments suitable for use with the grip element preferably have an elongated shape and are connected by a proximal end to the instrument holder. Accordingly, their orientation is specified by the orientation of their long direction. In order to avoid sideways bending of the wrist, the handle can be rotated about a handle axis that lies within the grip-element plane and is substantially parallel to the long direction of the handle. The handle axis is preferably disposed substantially in the center of the handle and runs through a connection site of the grip element, where the elongated handle and the instrument holder join one another. The elongated handle is preferably formed in such a way that the hand can grasp around it or enclose it altogether or only in part.

It often happens that a surgical instrument must be held in such a way that it tilts up or down. Regarding the directional terminology, the grip-element plane is again assumed to be vertical. So as to avoid bending of the wrist in this case as well, the handle is pivotable about an axis oriented substantially perpendicular to the plane of the grip element. Accordingly, this arrangement allows the angle enclosed by the handle and the instrument holder to be altered, and thus an upward or downward tilting of the surgical instrument can also be compensated.

Furthermore, the surgeon may be prevented, e.g. by other appliances on the operation table, from positioning his hand behind the elongated surgical instrument in such a way that he can guide the surgical instrument to the operation site in the desired orientation. This problem is alleviated by making it possible to shift the handle along a transposition line perpendicular to the long direction of the surgical instrument. Preferably the transposition line is disposed so as to be perpendicular to the grip-element plane. According to one preferred embodiment the handle can be rotated about the axis of handle rotation by as much as 60°, in particular by up to 450, out of a basic position. Preferably the handle can be rotated away from the basic position in both directions, by the same angle.

Preferably a basic position is provided in which the handle is disposed in the grip-element plane. According to one preferred embodiment the handle can be shifted along the transposition line by up to 10 mm away from its basic position. In particular, it can be provided that the handle can be shifted by this distance away from the basic position in both directions along the transposition line.

Preferably the grip element is designed so that various surgical instruments can be installed therein. In particular different tubular-shaft instruments with different tools at their distal end can then be simply exchanged. An embodiment of the invention that satisfies this requirement comprises in the instrument holder a receptacle within which a proximal end section of a surgical instrument can be fixed in position.

Another advantage is obtained when the tool of the surgical instrument can be adjusted to different orientations relative to the grip element. For this purpose the receptacle is to be constructed so that the surgical instrument can be fixed within the receptacle in various orientations. Here it should in particular be understood that the surgical instrument is fixed within the receptacle with its proximal end section in various rotational positions with respect to an axis of rotation corresponding to the long direction of the surgical instrument.

Furthermore, the grip element can also be provided for only one specific surgical instrument. In this case the instrument holder can be made integral with the surgical instrument. In particular when the instrument holder is constructed in one piece with part of the surgical instrument, it is impossible for the grip element to be separated from the surgical instrument, so that it cannot be used for a variety of surgical instruments. The advantage of such an arrangement is that the surgical instrument can have a relatively compact structure, so that it can more easily be cleaned.

Because it should be possible to position the surgical instrument at a desired operation site by applying force to the handle, there are preferably provided on the grip element retaining means by which the handle can be locked in a basic position as well as in at least one rotated, linearly shifted and/or outwardly pivoted position relative to the instrument holder, and hence also relative to the surgical instrument. This retention ensures that the movements carried out at the handle are also transmitted to the distal end of the surgical instrument. Preferably the handle can be locked by the retaining means in every position that it is possible for it to occupy relative to the instrument holder.

As discussed above, surgical instruments often comprise at their distal end a tool that can be moved by actuating an actuation element, or to which an electrical voltage can be applied by actuating an actuation element. Such an actuation element is preferably disposed on the instrument holder and/or the handle. If the actuation element is disposed on the handle, the transmission mechanism that transmits the initiated movement or signal to the tool at the distal end of the surgical instrument should be designed so that it permits rotation and/or linear movement of the handle. Preferably the actuation element is constructed as a pivoted lever. Then actuation is brought about, for example, by pulling the pivoted lever in the direction of the handle.

The advantages in accordance with the invention cited above are also obtained for a surgical instrument, in particular for a surgical tubular-shaft instrument, that comprises a grip element in accordance with the invention. A preferred arrangement of a surgical instrument is obtained when the surgical instrument is constructed in elongated form, comprises at its distal end a tool and at its proximal end the grip element, and furthermore the long direction of the surgical instrument lies substantially within the plane of the grip element. In particular, it is advantageous for the handle to extend away from the instrument holder at an angle to the long direction of the surgical instrument.

It is also advantageous to use a grip element in accordance with the invention for holding a surgical instrument, in particular for holding a surgical tubular-shaft instrument.

Advantages and useful features will further be evident from the following description of preferred exemplary embodiments with reference to the figures, wherein

### BRIEF DESCRIPTION OF THE DRAWINGS

FIG. 1 shows a perspective view of a grip element in accordance with the invention;

FIG. 2 shows a plan view from above of the grip element in FIG. 1;

FIG. 3 shows a schematic side view of another embodiment of a grip element in accordance with the invention within which a tubular-shaft instrument is held.

### DETAILED DESCRIPTION OF THE INVENTION

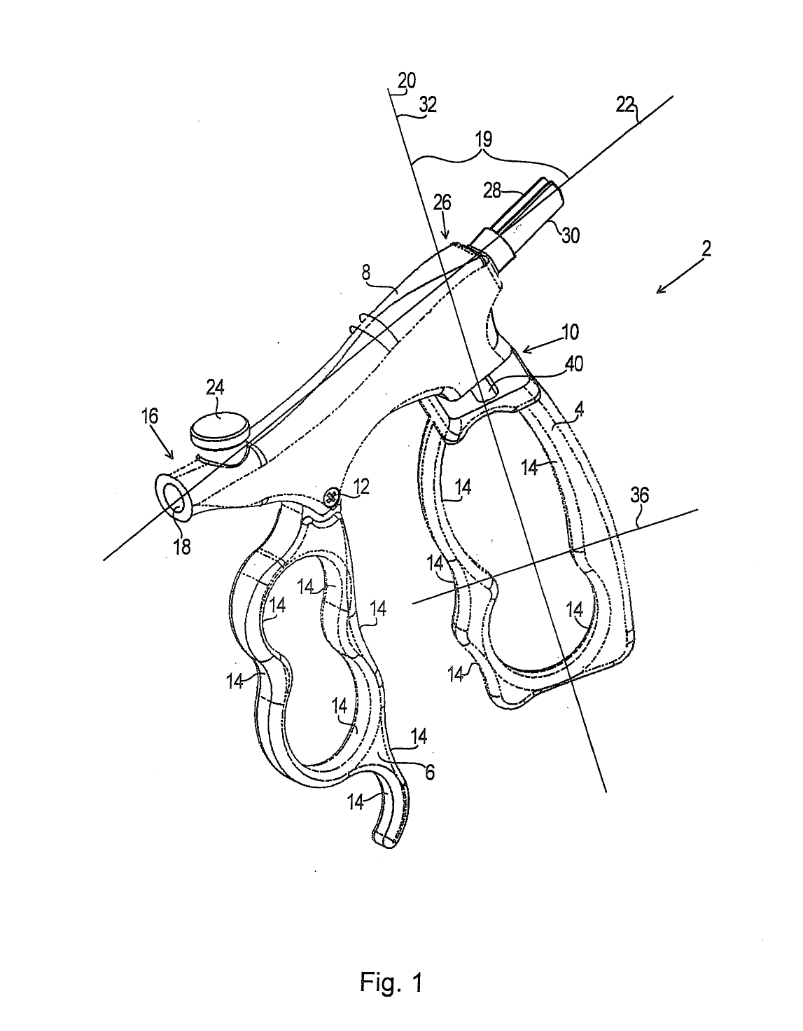

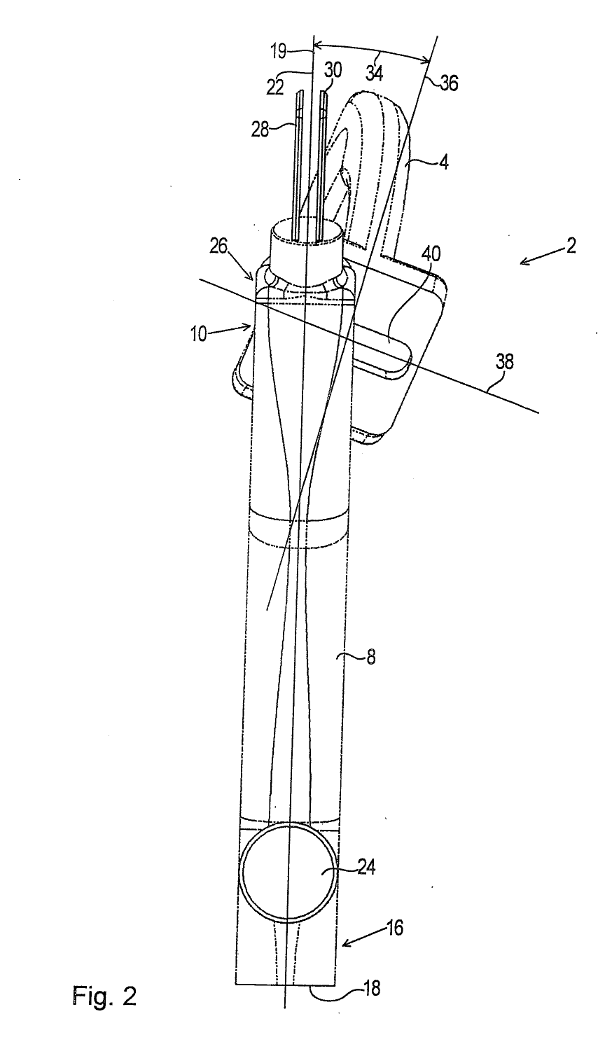

FIGS. 1 and 2 show a grip element 2 in accordance with the invention, with a handle 4, an actuation element 6 and an instrument holder 8. The handle 4 and the instrument holder 8 are joined at a connection site 10. The actuation element 6 is attached to the instrument holder 8 so as to be pivotable about an axis of rotation 12. In addition, on the handle 4 and the actuation element 6 there are provided a plurality of bearing surfaces 14 for parts of a surgeon's hands, so that it is possible to grip the handle 4 and operate the actuation element 6 with the hand in various positions.

The instrument holder 8 comprises at a distal end 16 a shaft receptacle 18, into which a proximal end section of a surgical instrument can be inserted. The shaft receptacle 18 shown here is designed to hold a tubular shaft in such a way that the proximal end of the tubular shaft can be fixed within the shaft receptacle 18 in various orientations. Accordingly, a tool disposed at the distal end of the tubular-shaft instrument can be oriented and fixed in various angular positions relative to a grip-element plane 19, which in a basic position of the handle 4 is defined by a long direction 20 of the handle 4 and an extent direction 22 of the instrument holder 8. The basic position of the handle 4 is characterized by the fact that the long direction 20 of the handle 4 and the extent direction 22 of the instrument holder 8 intersect and are not displaced transverse to one another. To fix the tubular shaft within the shaft receptacle 18, a retaining device 24 is provided at the shaft receptacle 18, by means of which the inside diameter of the shaft receptacle 18 can be made smaller. The retaining device 24 exerts a spring force that clamps the tubular shaft within the shaft receptacle 18.

There is additionally provided in the instrument holder 8 a transmission mechanism (not shown) through which a movement of the actuation element 6 can be transmitted to a tool of a tubular-shaft instrument held within the shaft receptacle 18. Thus pulling on the actuation element 6 can bring about an action of a tool on a tubular-shaft instrument held in the shaft receptacle 18.

Attached to a proximal end 26 of the instrument holder 8 are two electrical contacts 28, 30. In addition two electrical leads (not shown) that run within the instrument holder 8 are guided from the contacts 28, 30 to two corresponding contacts in the shaft receptacle 18, so that an electrical voltage can be applied to an appropriately constructed tubular-shaft instrument. In particular, a high-frequency alternating voltage can be applied between two tool parts of such a tubular-shaft instrument. A tubular-shaft instrument suitable for application of a high-frequency alternating voltage is, for instance, a bipolar forceps in which, when installed, each of the two jaws of the forceps is conductively connected to one of the electrical contacts 28, 30.

The following further explanation with reference to FIGS. 1 and 2 concerns the handle 4, which in accordance with the invention can be set into various positions relative to the instrument holder 8. In FIGS. 1 and 2 the handle 4 is shown in a rotated position, when it has been pushed out of the grip-element plane 19. The handle 4 can be rotated relative to the instrument holder 8 about a handle axis of rotation 32, which in the basic position of the handle 4 corresponds to the long direction 20 of the handle 4. As can be seen in FIG. 2, the angle 34 between a transverse direction 36 of the handle and the extension direction 22 of the instrument holder 8 can thus be varied.

In a basic position (not shown) the long direction 20 and transverse direction 36 of the handle are both within the grip-element plane 19. Oriented perpendicular to the transverse direction 36 of the handle, and perpendicular to the grip-element plane 19 when the handle 4 is in the basic position, is a transposition line 38 along which the handle 4 can be shifted relative to the instrument holder 8. When the handle 4 is shifted out of the basic position along the transposition line 38, the handle 4 and in particular its long direction 20 are displaced laterally, out of the grip-element plane 19.

The rotation and shifting of the handle 4 described above are implemented by a pin (not shown) disposed on the instrument holder 8, and an aperture 40 formed in the handle 4. The pin extends away from the instrument holder 8, within the grip-element plane 19 and perpendicular to the extent direction 22, passing through the aperture 40 in the handle 4. The aperture 40 is elongated and extends along the transposition line 38, so that the pin is held within the aperture 40 in such a way as to permit both rotation and shifting along the transposition line 38. The pin further comprises a thickening in a section behind the aperture 40, which prevents the handle 4 from being pulled away, for instance along the long direction 20 of the handle 4.

In addition there are provided at the connection site 10, where the pin passes into the aperture 40, retaining means (not shown) that keep the handle 4 fixed in a desired position relative to the instrument holder 8. Starting from the basic position, the illustrated grip element permits the handle 4 to be rotated in both directions about the handle axis 32, preferably by up to 600. Furthermore, this arrangement enables the handle 4 to be shifted along the transposition line 38 away from the basic position of the handle 4 in both directions, preferably by up to 10 mm.

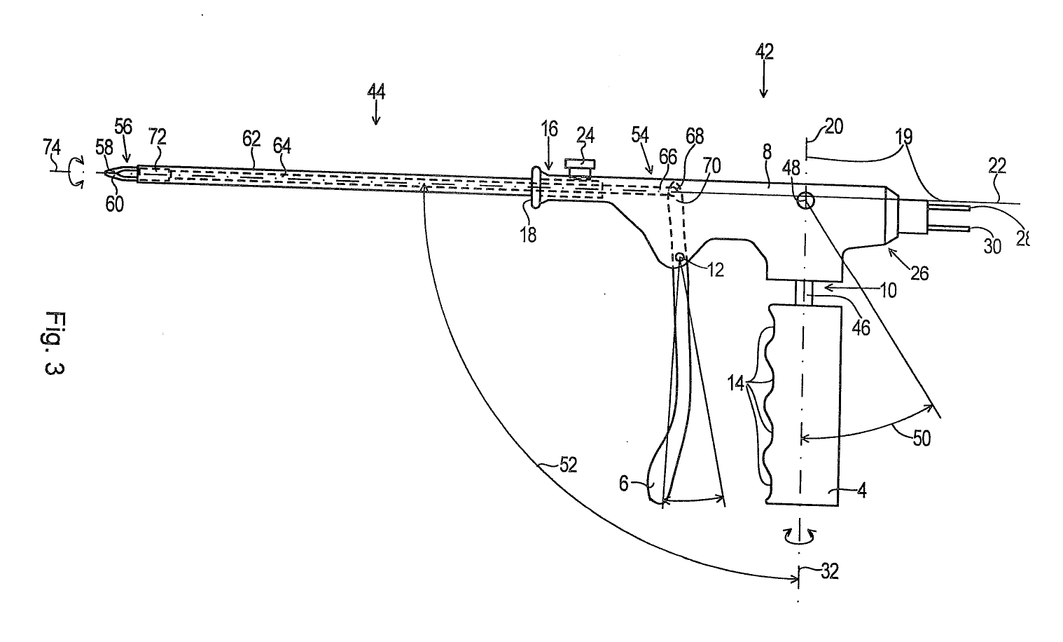

FIG. 3 is a schematic drawing of another embodiment of a grip element 42 in accordance with the invention, with a tubular-shaft instrument 44 designed as forceps installed therein. As in the first embodiment, this grip element comprises a handle 4, an actuation element 6, an instrument holder 8, electrical contacts 28, 30, a shaft receptacle 18 and a fixation screw 24. In the case of the grip element 42 shown in FIG. 3, again, a pin 46 extends from the instrument holder 8 into an aperture (not shown) in the handle 4, so that as in the first embodiment the handle can be both rotated relative to the instrument holder 8, about an axis of handle rotation 32, and shifted transverse to a grip-element plane 19.

The pin 46 is held within the instrument holder 8 in such a way that it can be pivoted about an axis 48 oriented perpendicular to the grip-element plane 19. The handle 4 can be pivoted within the grip-element plane 19 in the direction indicated by the arrow 50, out of a basic position in which the angle 52 enclosed between the long direction 20 of the handle 4 and the extent direction 22 of the instrument holder 8 amounts to 90°. According to this embodiment, therefore, there is the additional possibility of varying the angle 52 enclosed between handle 4 and instrument holder 8.

FIG. 3 shows schematically a transmission mechanism 54 by means of which a movement of the actuation element 6 can be transmitted to a tool 56, which in the present case is formed by two forceps jaws 58, 60. Within a tubular shaft 62 of the tubular-shaft instrument 44 is disposed a push/pull rod 64, the proximal end 66 of which can be connected to an upper section 70 of the actuation element 6, by means of a ball-type rapid-action closure 68. By actuating the actuation element 6, the push/pull rod 64 is moved relative to the tubular shaft 62. Furthermore, near the tool 56 within the tubular shaft 62 there is disposed a conversion device 72, by means of which the relative movement between tubular shaft 62 and push/pull rod 64 is converted to a relative movement between the forceps jaws 58, 60, so that moving the actuation element 6 causes opening or closing of the forceps jaws 58, 60.

The tubular-shaft instrument 44 can furthermore be rotated within the shaft receptacle 18 about its long direction 74, which is parallel to the extent direction 22 of the instrument holder 8, and can be retained in a desired orientation by tightening the fixation screw 24. Accordingly, the orientation of the tool 56 relative to the grip-element plane 19 can be adapted to suit the present application. The invention is not limited to the embodiments shown as examples in FIGS. 1 to 3. Instead the invention results from an expert overall consideration of the claims, the description, the exemplary embodiments and the variants mentioned below, which are intended to provide a person skilled in the art with indications of additional alternative embodiments.

In particular, the axis of handle rotation, the transposition line, the long direction of the handle, the direction of extent of the instrument holder and the pivot axis can be oriented with respect to one another in ways different from those shown in FIGS. 1 to 3. Furthermore, additional axes of rotation and/or transposition lines can also be provided, to permit more movements of the handle relative to the instrument holder.

The connection between the handle and the instrument holder, which permits rotation and linear shifting of the handle relative to the instrument holder, is not limited to the illustrated arrangement of pin and aperture. In particular, it is also possible for the handle to comprise a pin and the instrument holder, an aperture for the pin. Furthermore, a ball joint between handle and instrument holder can also be provided, which permits at least a rotary movement of the handle about a handle axis lying substantially within the grip-element plane, and a pivoting movement of the handle about a pivot axis oriented transverse to the grip-element plane.

For transmission of a movement of the actuation element to a tool belonging to the surgical instrument, in addition to the illustrated arrangement of tubular shaft and push/pull rod that can be moved relative thereto and is or can be mechanically connected to the actuation element a large number of other transmission mechanisms are also known, which can likewise be converted for use in a grip element in accordance with the invention.

The configuration of the handle, the actuation element and the instrument holder shown here is not crucial for embodying the invention, so that here again additional possibilities for variation exist. In particular the actuation element can equally well be constructed as a sliding switch, pressure switch or the like. For positioning of the electrical contacts, too, there are other possible variations.

Furthermore, in FIGS. 1 to 3 a shaft receptacle is shown that is designed to receive a tubular section of a tubular-shaft instrument. However, another kind of receptacle can be provided at the instrument holder, within which a proximal end section of a surgical instrument can be fixed. Similarly, the means for fixing the surgical instrument in the receptacle need not be implemented as a fixation screw, but can take the form of another kind of clamping or fixing means.

### LIST OF REFERENCE NUMERALS

2 Grip element
4 Handle
6 Actuation element
8 Instrument holder
10 Connection site
12 Axis of rotation
14 Bearing surface
16 Distal end
18 Shaft receptacle
19 Plane of grip element
20 Long direction (of the handle)
22 Extent direction (of the instrument holder)
24 Fixation screw
26 Proximal end
28, 30 Electrical contacts
32 Axis of handle rotation
34 Angle
36 Transverse direction of handle
38 Transposition line
40 Aperture
42 Grip element
44 Tubular-shaft instrument
46 Pin
48 Pivot axis
50 Arrow showing direction
52 Angle
54 Transmission mechanism
56 Tool
58, 60 Forceps jaws
62 Tubular shaft
64 Push/pull rod
66 Proximal end
68 Ball-type rapid closure
70 Upper section
72 Conversion device
74 Long direction

1. Grip element for a surgical instrument, comprising:
a handle; and
an instrument holder for the surgical instrument, said handle being adapted such that it moves by at least one of rotation and linear motion relative to the instrument holder.

2. Grip element according to claim 1, defining a grip-element plane within wich said handle and said instrument holder rotation about which said handle is adapted to rotate that is situated within said grip-element plane and that is substantially parallel to the long direction of said handle.

3. Grip element according to claim 1, defining a grip-element plane within which said handle and said instrument holder predominantly extend, and a transposition line that is oriented substantially perpendicular to said grip-element plane along which transposition line said handle is adapted to be shifted linearly relative to said instrument holder.

4. Grip element according to claim 1, defining a a grip-element plane along which said handle and said instrument holder predominantly extend, and wherein said handle is adapted to rotate about a pivot axis that is substantially perpendicular to said grip-element plane.

5. Grip element according to claim 2, wherein said to handle is adapted to rotate about said axis of rotation in both directions of rotation by up to 60° away from a basic position.

6. Grip element according to claim 3, wherein said handle is adapted to move linearly out of a basic position, in which said handle is situated within said grip-element plane, along said transposition line in both directions, by up to 10 mm.

7. Grip element according to claim 1, wherein said instrument holder defines a receptacle within which a proximal end section of a surgical instrument can be fixed in a plurality of orientations.

8. Grip element according to claim 1, wherein said instrument holder is constructed integral with said surgical instrument.

9. Grip element according to claim 1, comprising a retaining means to fix said handle in a basic position and in at least one rotated, linearly shifted and/or outwardly pivoted position relative to said instrument holder.

10. Grip element according to claim 1, wherein at least one of said instrument holder and said handle comprises an actuation element for actuating said surgical instrument.

11. Grip element according to claim 10, wherein said actuation element comprises a pivoted lever.

12. Surgical instrument with a grip element comprising a handle and an instrument holder for the surgical instrument, wherein said handle is adapted such that it moves by at least one of rotation and linear motion relative to the instrument holder.

13. Surgical instrument according to claim 12, that has an elongated configuration and that has a tool disposed at its distal end and said grip element disposed at its proximal end such that a long direction of said surgical instrument lies substantially within a grip-element plane within which said handle and said instrument holder predominantly extend, and such that said handle slants away from said instrument holder at an angle to said long direction of the surgical instrument.

14. Use of a grip element according to claim 1 to hold a surgical instrument.

15. Grip element as claimed in claim 1, adapted for use with a tubular shaft surgical instrument.
